(19) United States
(12) Patent Application Publication
(10) Pub. No.: US 2025/0246261 A1
(43) Pub. Date: Jul. 31, 2025

(54) PROTEIN INVERSE FOLDING METHOD BASED ON MULTI-MODAL PRETRAINED LARGE MODEL

(71) Applicant: The Chinese University of Hong Kong, Hong Kong (CN)

(72) Inventors: Pheng Ann HENG, Hong Kong (CN); Guangyong CHEN, Hangzhou (CN); Jiezhong QIU, Hangzhou (CN); Hanqun CAO, Hong Kong (CN); Junde XU, Hangzhou (CN)

(21) Appl. No.: 18/425,749

(22) Filed: Jan. 29, 2024

(51) Int. Cl. G16B 15/20; G16B 30/20; G16B 40/20

(52) U.S. Cl. CPC G16B 15/20; G16B 30/20; G16B 40/20

(57) ABSTRACT

The subject invention pertains to a novel framework, ProteinAligner, that achieves the latent representations alignment of large pretrained model's prior knowledge. ProteinAligner fine tunes the autoregressive protein language models by without unfreezing any pretrained weight, connecting cross-modality deep latent features by training lightweight modules. Experiments validate the effectiveness of the provided alignment framework on open-source protein datasets according to perplexity of generation. The results demonstrate the proficiency of the provided tuning technique and the pretrained large models' impressive capability of protein sequence generation. Pretrained protein models in a novel framework ensure high-quality protein modeling and novel generation with high diversity. An alignment module between pretrained structure models and pretrained sequence models establishes the structure-sequence connection with limited data.

### BACKGROUND OF THE INVENTION

Computational protein design, known as protein inverse folding, has significant utility for protein functional design and drug discovery, aiming at creating novel foldable protein sequences with high diversity, enabling generation of folded protein structures with varieties of biological functions [17, 23]. With the aid of deep learning-based approaches, protein inverse folding enables the creation of more efficient enzymes [28, 26], improved protein-based therapeutics [4], and engineered proteins for industrial purposes like biofuels production and environmental remediation [27, 5].

Traditional machine learning techniques, while useful in addressing protein inverse folding, come with inherent limitations [15, 2]. They frequently require extensive data sets for training and their ability to accurately predict protein folding or effectively design novel, functional, and foldable proteins can be inconsistent [21, 25]. To investigate the long-range dependencies and dynamic interactions among protein residues, traditional deep learning-based approaches for protein inverse folding employ Graph Neural Network (GNN). The GraphTrans and StructGNN [12] models encode structural information as node and edge embeddings, and iteratively decode the node embeddings into protein sequences. The conditional protein language model captures both local dependencies and long-range interactions. The advent of deep learning, however, has revolutionized protein design. Powerful algorithms, proficient in interpreting intricate patterns within data, have bolstered the designability of proteins [3, 31, 13]. They facilitate the generation of protein sequences that fold as intended, thereby elevating the efficiency and precision of protein design. Furthermore, deep generative modeling has catalyzed the creation of novel proteins with a variety of functions, signifying a major breakthrough in the fields of biochemistry and drug discovery [23, 26].

Although being effective, one of the main challenges of deep-learning-based methods is scarce experimental protein data for training [22, 10, 14]. Due to the complexities of data acquisition, intricate data processing, and the vast diversity of proteins, the limited availability of high-quality protein sequence-structure paired data hinders real-world applications of protein inverse folding with deep neural networks [23]. Small-scale training data only enables lightweight deep-learning models to ensure accurate predictions as well as to avoid overfitting. Thus, current approaches for computational protein design rely on generative graph modeling, including GVP [13], ESM-IF [11], ProteinMPNN [3], and PiFold [9]. By enhancing the graph embedding with an additional scalar embedding based on dihedral angles, GVP [13] adheres to

the similar encoder-decoder pattern based on inter-residual message-passing and proposes a

model quality assessment scheme for best-candidate selection. GCA [29] proposes global local context interaction modules viewing from the lens of language modeling to generate protein sequence by direct mappings. AlphaDesign [8] presents a simplified graph encoder and a constraint aware decoder based on GVP [13]. By training with data from AlphaFoldDB [14], the framework captures effective constraints and accurate interactions among graph nodes. The ProteinMPNN [3] further capitalizes on the benefits of an auto-regressive encoding-decoding scheme and message passing updating techniques. It enhances the structural position information of the Oxygen atom and pre-calculated Cβ atom into protein structural representations. In addition, ProteinMPNN [3] extends computational design to multi-chain proteins and multi-scale noise adaptation. By introducing virtual atoms and backbone dihedrals, PiFold [9] improves the traditional encoding decoding framework into equal-dimension protein encoders, achieving a 70-fold acceleration compared to ProteinMPNN [3]. However, while effective in designing novel protein sequences, the diversity of generated sequences is limited by the small scale of training data. These methods achieve effective encoding and decoding for sequence design, depending on hierarchical usage of protein structure information and dynamic interaction capturing by message-passing.

However, lightweight models suffer from many drawbacks. With regard to protein modeling and designability, a limited amount of data fails to support global context learning for autoregressive models based on protein language [29]. Moreover, complicated human-designed features are insufficient for robust structure representations, leading to underutilized information [3, 8, 9]. In terms of protein sequence generation, fraction access to the universal protein distribution locks the capability of vast novel protein generation, impeding cross-distributional search of potential protein sequences [30]. Leveraging the highly accurate protein folding of AlphaFold2, ESM-IF [11] trains a large-scale inverse folding framework based on GVP [13]. It utilizes approximately 16,000 high-quality protein sequence structure data from CATH [22] and about 12 million data with noise obtained from AlphaFold2 [14]. ESM-IF [11] achieves high

recovery of protein sequences but incurs a high computational cost. LM-Design [31] conducts inverse folding by utilizing effective embeddings from pretrained protein models and recovers design sequences through conditional mask predicting. Although LM Design [31] efficiently

solves limited data problems by fine-tuning ESM models [24], the current approach does not promote natural protein sequences that exhibit dynamic lengths and differences due to its masked generation scheme.

Structure-based protein is becoming increasingly necessary as a powerful tool for exploring novel proteins with specific functionality. Related art data-driven deep learning approaches have provided limited achievement in high-fidelity inverse folding by generative learning techniques. Despite these successes, computational design confronts the challenges of limited amount of experimentally selected data, which leads to deficiencies in the generative power of deep neural network and low generalization.

### BRIEF SUMMARY OF THE INVENTION

Computational protein design, also known as protein inverse folding, aims to provide novel and diverse foldable protein sequences based on given protein backbone structures. Related art deep learning-based approaches are limited by the amount of high-quality training data, leading to poor generalizability and restricted application value. To address this issue, embodiments of the subject invention provide pretrained protein models into a framework to ensure high-quality protein modeling and novel generation with high diversity. Embodiments provide an alignment module between pretrained structure models and pretrained sequence models to establish a beneficial structure-sequence connection with limited data.

Embodiments of the subject invention apply novel and advantageous sequence-to-sequence language models as autoregressive generators, which are useful for valuable sequence exploration. Related art approaches trained on small-scale data exhibit weak generalization capabilities, which limits the real-world applications for protein exploration and functional sequence design [9, 13].

To address this issue, embodiments utilize large pretrained protein models from both modalities [6]. Large pretrained models (e.g., trained on 109 high-fidelity data of single modality, alternatively at least 100, alternatively at least 109, alternatively 100 to 120, alternatively 90 to 130 data of single modality, including increments, combinations, and ranges of any of the foregoing) acquire strong prior knowledge of the universal distribution of protein, leading to more accurate protein structure and sequence modeling as well as higher generalizability [24, 20, 19]. Getting closer to the nature of protein modeling, applying combined knowledge of pretrained models exhibits wider possibilities for obtaining protein sequences with higher designability and generalizability [7, 11]. However, employing large pretrained models requires high computational cost, including large amount of trainable parameters and high dimension of representations. More importantly, aligning deep latent features with cross-modality data requires millions of paired data in the field of vision-language fusion, which brings huge challenges for small-scale data alignment [18, 16, 1]. Thus, only one work has been developed to this issue. LM-Design utilizes the structure embeddings from pretrained structure networks, conducting sequence generation by pretrained ESM model with masked language modeling (MLM) manner of generation [31]. While LM-Design achieves higher performance than the all pretrained structure encoders for protein inverse folding, its application for sequence generation is limited by the nature of MLM since it only enables equal-length design. Thus, to achieve alignment based on larger pretrained models (e.g., 6.4B param) and autoregressive sequence generation with the same amount of data [20], embodiments of the subject invention advantageously provide a novel and useful inverse folding framework, referred to herein as ProteinAligner. The ProteinAligner module achieves efficient billion-level tuning and lightweight deep alignment by simple cross-attention design between sequence and structure embeddings. In experiments, an embodiment comprising ProteinAligner is proved to achieve effective tuning of pretrained protein large language models (LLM) and cross-modality alignment. The provided framework presents excellent designability based on perplexity of 3.00 on CATH4.2 dataset and highly diverse and novel generation.

Embodiments provide a novel protein inverse folding framework base on structure-sequence latent feature alignment as well as autoregressive sequence generation. The provided framework is flexible for advanced pretrained structure encoders and sequence decoders.

The provided ProteinAligner design involves highly efficient design for aligning large pretrained models with limited amount of data. The one-stage alignment without tuning pretrained models is advantageously easy to implement and computation friendly.

Experiments have shown that the provided framework based on the large language models and large structure encoders exhibits high designability for protein sequences as well as diversely novel designs. Embodiments comprising ProteinAligner demonstrate not only effective LLM-tuning, but also high performance on sequence-structure alignment.

### BRIEF DESCRIPTION OF THE DRAWINGS

FIG. 1 Illustrates a protein design system 10 comprising 100, 200, and 300. according to an embodiment of the subject invention incorporating other related art graph-based computational protein design methods to provide improved overall systems and methods. Graph-based approaches consist of two generation strategies, including autoregressive generation [3] and one-shot generation [9]. The structure-sequence matching is conducted through the Encoder-Decoder framework. In ProteinAligner, embodiments apply the pretrained encoders for protein structure expression, with frozen parameters. Also, autoregressive pretrained protein sequence decoders [20] are frozen as protein sequence autoregressive generators. The deep learning-based latent representation alignment model implements the latent representation alignment.

### DETAILED DISCLOSURE OF THE INVENTION

Embodiments of the subject invention provide three main advantages: (1) Embodiments utilize pretrained protein models to enhance featurization of protein, and therefore to build accurate mappings between protein structure and protein sequence. (2) Embodiments advantageously employ a lightweight module for latent feature alignment that improves the efficiency of fine tuning large models and achieves beneficial results with limited data. (3) Embodiments can generate protein sequences with higher novelty and diversity. Moreover, the generated protein sequences have higher designability.

Embodiments provide a framework for computational protein sequence design and protein inverse folding that solves the above noted problems related to designing novel, diverse, and foldable protein sequences based on the protein structures. The designed sequences have high-fidelity according to perplexity. In the real-world applications, embodiments can be used to explore functional sequences with the same structure, and functional protein search. Also, with the aid of large pretrained models, the generated instances can provide higher diversity and novelty, which reduces the cost of human filtering and selection.

The provided pipeline or Multi-Modal Alignment Module 100 according to certain embodiments is designed for protein inverse folding and computational protein sequence design. It can be a sophisticated system comprised of several components, each of which plays a significant role in the overall process. The first component is a pretrained deep learning-based encoder configured for protein structure latent expression in a high dimensional space 102. This model is trained on a vast amount of data to learn the three-dimensional structures of proteins. In one embodiment, three types of pretrained protein structure models are used, which are ProteinMPNN, PiFold, and ESM-IF. ProteinMPNN and PiFold are trained on CATH4.2 dataset, which has 18006 training instances. For ESM-IF, it uses augmented data from AlphaFold Database. The estimated amount of training data is 12 million structures. It is designed to output latent structure embeddings in a high dimensional space 111. This means that the model can generate a compressed representation of a protein's structure, which can then be used for further analysis or to feed into other models. The second component of the pipeline is an autoregressive pretrained protein sequence decoder 104. This model leverages protein language modeling techniques to generate protein sequences automatically. Autoregressive models are powerful tools in machine learning that are capable of predicting a sequence of outputs based on a sequence of inputs. In this case, the model is trained to predict the next amino acid in a protein sequence based on the preceding sequence of amino acids. The third component is a deep learning-based latent representation alignment model 103. This model is trained to learn the connections between the latent representations of protein sequences and protein structures. The alignment model is a crucial part of the pipeline as it bridges the gap between the sequence and structure of proteins. It does this by learning to map the latent representations of protein sequences to their corresponding protein structures. This is a challenging task due to the complex nature of proteins, but a successful alignment model can provide valuable insights into the relationship between a protein's sequence and its structure. In certain embodiments, the provided pipelines according to certain embodiments of the subject invention offer a comprehensive solution for protein inverse folding and computational protein sequence design. Embodiments leverage advanced deep learning techniques to learn protein structures, generate protein sequences, and align these sequences with their corresponding structures.

Related art methods learn mappings from protein structures to protein sequences from scratch. However, they train the models with only about 20 thousand data points, which causes a low quality of protein data representation. Furthermore, compared to embodiments of the subject invention, the limited training data leads to low generalizability, low diversity, and low novelty of protein sequence generation. Embodiments of the subject invention advantageously apply strong prior knowledge from pretrained protein large language models, and the high-fidelity representation of protein boosts high-quality mappings from protein structures to protein sequences. Embodiments have achieved unmatched levels of performance on multiple protein sequence design benchmarks.

The inventors have evaluated the provided framework on 5 metrics, including: (1) perplexity on generated sequences; (2) sequence recovery on generated sequences; (3) DEDAL Score (Deep Embedding Differential Alignment Score) on generated sequences; (4) self-consistency RMSD and self-consistency TM-Score between original protein structures and folded structures on generated sequences; and (5) bioinformatics features appearance between original protein structures and folded structures on generated sequences. Provided metrics (1), (2), and (4) are common in this field; (3) is a new metric for evaluating protein sequence recovery (Llinares-López, et al. Deep embedding and alignment of protein sequences. Nat Methods 20. 104-111 (2023). https://doi.org/10.1038/s41592-022-01700-2, https://www.nature.com/articles/s41592-022-01700-2); an (5) is a common metric in bioinformatics analysis.

This innovation can assist biology researchers in identifying protein sequences capable of conforming to a desired structure, a crucial aspect in several applications like medicinal formulation, functional enzyme, and functional peptide engineering. Based on this innovation, researchers in biology can expediently reduce the set of potential sequences using computer-based studies, focusing their experimental validation only on the sequences with the highest confidence. This approach significantly reduces their workload, controls experimental cost, and accelerates the process of protein discovery.

Embodiments of the subject invention address the technical problem of traditional machine learning techniques requiring extensive data sets for training and their ability to accurately predict protein folding or effectively design novel, functional, and foldable proteins being inconsistent. This problem is addressed by providing (1) pretrained protein models to enhance featurization of protein, (2) a lightweight module for latent feature alignment, and (3) the ability to generate protein sequences with higher novelty and diversity; in which a deep learning method applying a combination of these advanced techniques is utilized to (1) build accurate mappings between protein structure and protein sequence, (2) improve the efficiency of fine tuning large models and achieve beneficial results with limited data, and (3) generate protein sequences that have higher designability.

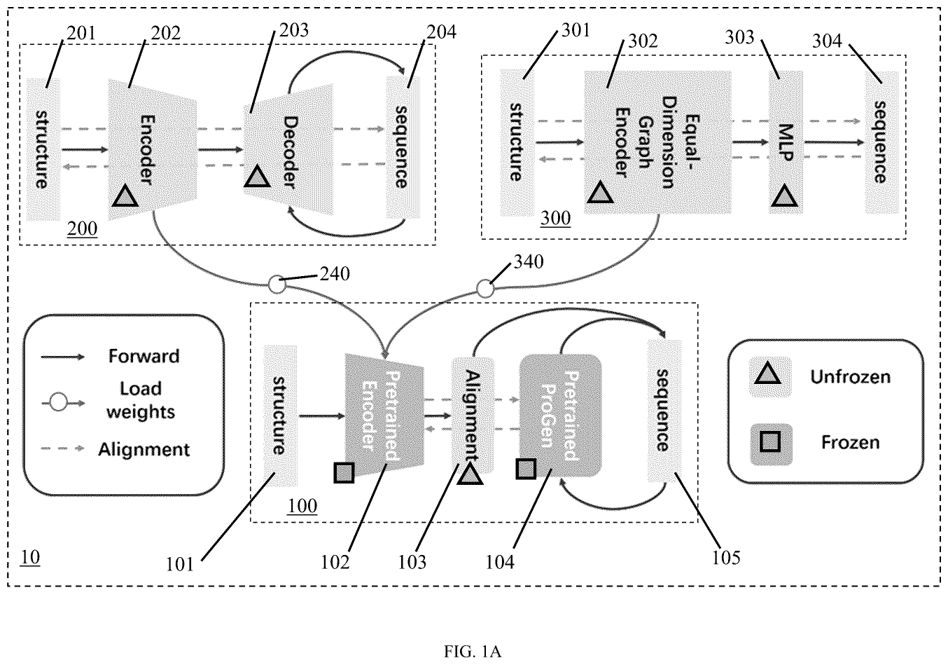

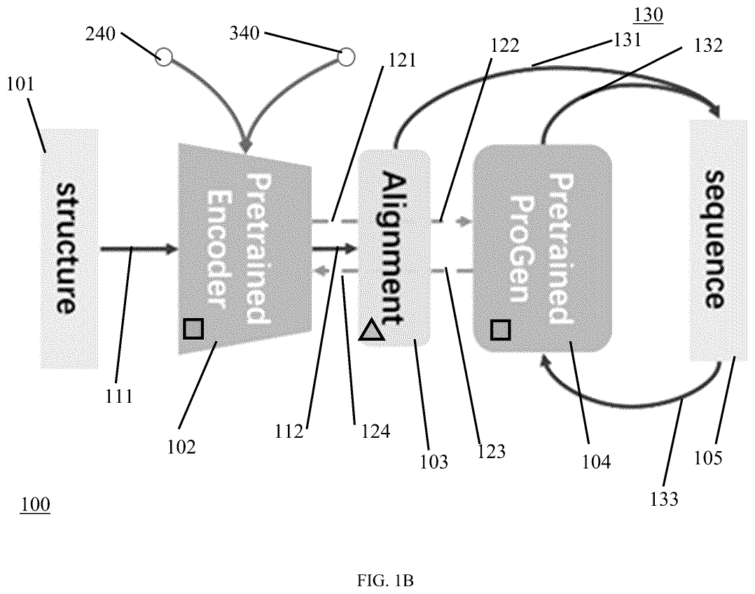

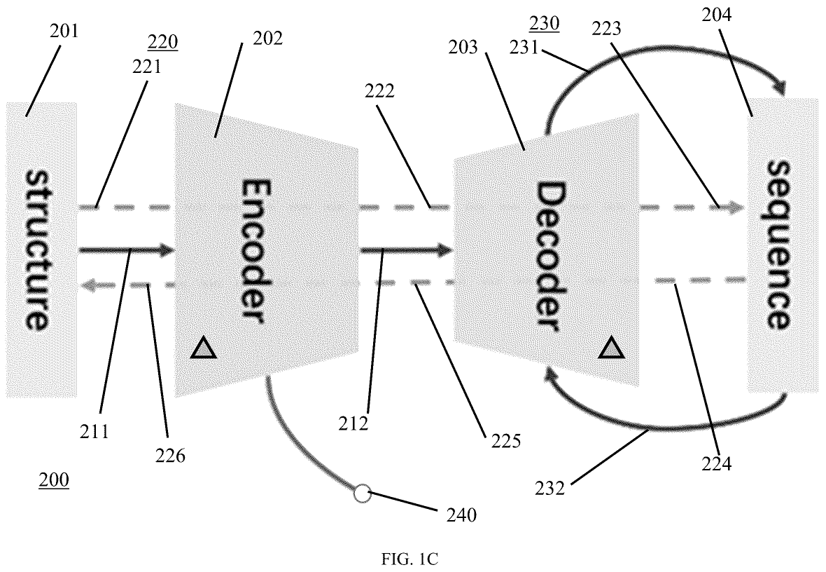

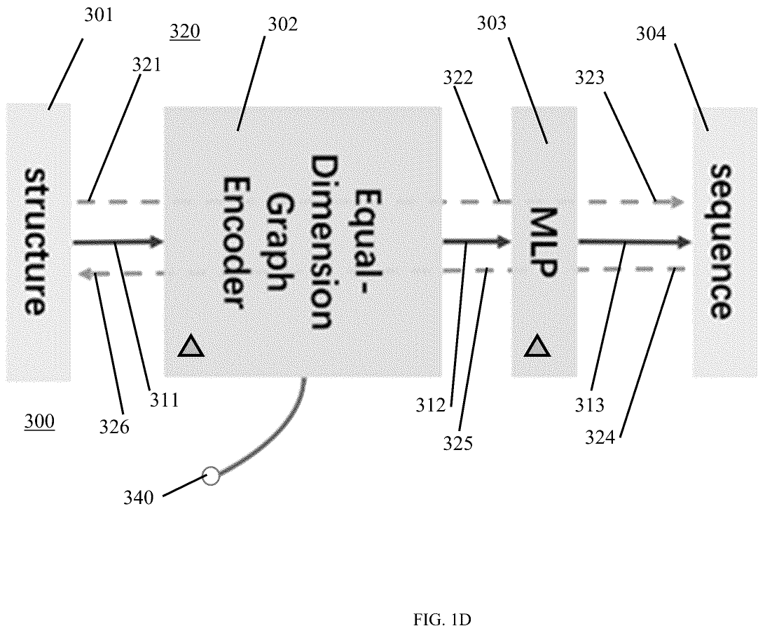

Turning now to the figures, FIG. 1A illustrates a protein design system 10 (comprising all elements within the outermost broken line box surrounding FIG. 1A) according to an embodiment of the subject invention incorporating other related art graph-based computational protein design methods to provide improved overall systems and methods. Graph-based approaches consist of two generation strategies, including autoregressive generation [3] and one-shot generation [9]. The structure-sequence matching is conducted through the Encoder-Decoder framework. In ProteinAligner, embodiments apply the pretrained encoders for protein structure expression, with frozen parameters. Also, autoregressive pretrained protein sequence decoders [20] are frozen as protein sequence autoregressive generators. The deep learning-based latent representation alignment model implements the latent representation alignment.

Multi-modal alignment module 100 (as shown in FIGS. 1A and 1B) feeds input structure 101 (via solid straight arrow 111) through pretrained encoder 102 and into protein aligner 103. A forward process (solid curved arrow 116) connects protein aligner 103 to output sequence 105. A feedback loop process (pair of solid curved arrows 117 and 118) connects autoregressive pretrained protein sequence decoder (ProGen) 104 to output sequence 105. Alignment processes (via broken-line arrows 112, 113, 114, and 115) feed forward from pretrained encoder 102, into protein aligner 103, and then to autoregressive pretrained ProGen 104 and then backward from autoregressive pretrained ProGen 104, through protein aligner 103, and into pretrained encoder 102.

Autoregressive graph-based generator 200 (as shown in FIGS. 1A and IC) feeds input structure 201 (via solid straight arrows 211 and 212, respectively) through encoder 202 and into decoder 203. A feedback loop process (pair of solid curved arrows 218 and 219) connects decoder 203 to output sequence 204. Alignment processes (via broken-line arrows 212, 213, 214, 215, 216, and 217) feed forward and back through each of elements 201, 202, 203, and 204.

One-shot graph-based generator 300 (as shown in FIGS. 1A and ID) feeds input structure 301 (via solid straight arrows 311, 312, and 313, respectively) through equal dimension graph encoder 302 and multi layer perceptron (MLP) 303, to provide sequence 304. Alignment processes (via broken-line arrows 313, 314, 315, 316, 317, and 318) feed forward and back through each of elements 301, 302, 303, and 304.

Encoder 202 and Equal-Dimension Graph Encoder 302 are different types of protein structure encoders and each, respectively, encodes protein structures with different network structures.

In certain embodiments, the Encoder 202 and the Equal-Dimension Graph Encoder 302 represent different approaches for encoding protein structures, each rooted in different methodologies for designing protein sequences. The Encoder 202 employs an auto-regressive generation approach with next-token prediction. As a result, the Encoder 202 needs to adjust its structure together with the decoder 203. On the other hand, the Equal-Dimension Graph Encoder 302 and multi-layer perceptron (MLP) 303 adopt a one-shot protein sequence generation strategy, sampling sequence through single round of inference. Consequently, the equal-dimensional encoder is better suited for this type of generation. In summary, the structural differences between the Encoder 202 and the Equal-Dimension Graph Encoder 302 originate from different patterns of protein sequence design, resulting in different network structures—mainly in input and output dimensions—and varying shapes of the output.

Throughout the figures, arrows indicate information and/or process flows. For example, black arrows 111, 112, 131, 132, 133, 211, 212, 231, 232, 311, 312, and 313 indicate a respective forward process of the model, blue arrows 240 and 340 indicate the loading of respective pretrained weight(s) from specific modules, and (red/orange, broken line) alignment arrows 121, 122. 123. 124. 221. 222. 223. 224. 225. 226. 321. 322. 323. 324. 325, and 326 indicate establishing respective connections in deep learning models to learn the mappings between specific data.

TABLE 1
Selected elements of certain embodiments of the subject invention.

| Number | Description | Comment |
|---|---|---|
| 10 | Multi-modal pretrained | Embodiments can comprise all elements of 100, 200, |
| | protein language model based | and 300. |
| | protein design system | |
| 100 | Multi-modal alignment | Embodiments can comprise all elements 101-133. |
| | pipeline | |
| 101 | Protein structure | Input |
| 102 | Pretrained Encoder | pretrained deep learning-based encoder configured |
| | | for protein structure latent expression in a high |
| | | dimensional space |
| 103 | ProteinAligner | deep learning-based latent representation alignment |
| | | model |
| 104 | Pretrained ProGen | autoregressive pretrained protein sequence decoder |
| 105 | Protein sequence | output |
| 111 | Forward process | Model inference |
| 112 | Forward process | Model inference |
| 121 | Alignment process | to learn the mappings between specific data |
| 122 | Alignment process | to learn the mappings between specific data |
| 123 | Alignment process | to learn the mappings between specific data |
| 124 | Alignment process | to learn the mappings between specific data |
| 131 | Forward process | Model inference |
| 132 | Forward process | Model inference |
| 133 | Forward process | Model inference |
| 200 | Autoregressive Graph-based | Embodiments can comprise all elements 201-240. |
| | generation module | |
| 201 | Protein structure | input |
| 202 | protein structure encoder | Auto-regressive generation based protein encoder |
| 203 | protein sequence decoder | Auto-regressive generation based protein decoder |
| 204 | Protein sequence | output |
| 211 | Forward process | Model inference |
| 212 | Forward process | Model inference |
| 221 | Alignment process | to learn the mappings between specific data |
| 222 | Alignment process | to learn the mappings between specific data |
| 223 | Alignment process | to learn the mappings between specific data |
| 224 | Alignment process | to learn the mappings between specific data |
| 225 | Alignment process | to learn the mappings between specific data |
| 226 | Alignment process | to learn the mappings between specific data |
| 231 | Forward process | Model inference |
| 232 | Forward process | Model inference |
| 240 | Encoder weights | loading of pretrained autoregressive encoder |
| | | weight(s) |
| 300 | One-shot graph-based | Embodiments can comprise all elements 301-340. |
| | generator | |
| 301 | Protein structure | input |
| 302 | Protein structure encoder | One-shot generation-based Equal-Dimension Graph |
| | | Encoder |
| 303 | Protein sequence decoder | multi layer perceptron (MLP) or One-shot |
| | | generation-based Equal-Dimension Decoder |
| 304 | Protein sequence | output |
| 311 | Forward process | Model inference |
| 312 | Forward process | Model inference |
| 313 | Forward process | Model inference |
| 321 | Alignment process | to learn the mappings between specific data |
| 322 | Alignment process | to learn the mappings between specific data |
| 323 | Alignment process | to learn the mappings between specific data |
| 324 | Alignment process | to learn the mappings between specific data |
| 325 | Alignment process | to learn the mappings between specific data |
| 326 | Alignment process | to learn the mappings between specific data |
| 340 | Equal-Dimension Graph | loading of pretrained one-shot encoder weight(s) |
| | Encoder weights | |

In certain embodiments, the One-Shot and Auto-regressive graph models outputs can be used solely as the training input for embodiment of the protein structure model framework. They can be regarded as the load-weighted model for producing the source of the provided protein structure model's input.

The load weights are necessarily derived from specific datasets. For example, ProteinMPNN and PiFold can be on CATH4.2 dataset, and ESM-IF can be on CATH4.3 dataset. In embodiments that train ProteinAligner on CATH4.2, then ProtienMPNN or PiFold can be used as pretrained structure encoders.

In certain embodiments, the One-Shot and Auto-regressive models are utilized to provide pre-loaded tools for producing inputs of the provided protein structure model framework.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting essentially of” or “consists essentially of” indicate that the claim encompasses embodiments containing the specified materials or steps and those that do not materially affect the basic and novel characteristic(s) of the claim. Use of the term “comprising” contemplates other embodiments that “consist of” or “consisting essentially of” the recited component(s).

When ranges are used herein, such as for dose ranges, combinations and subcombinations of ranges (e.g., subranges within the disclosed range), specific embodiments therein are intended to be explicitly included. When the term “about” is used herein, in conjunction with a numerical value, it is understood that the value can be in a range of 95% of the value to 105% of the value, i.e., the value can be +/−5% of the stated value. For example, “about 1 kg” means from 0.95 kg to 1.05 kg.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

A greater understanding of the embodiments of the subject invention and of their many advantages may be had from the following examples, given by way of illustration. The following examples are illustrative of some of the methods, applications, embodiments, and variants of the present invention. They are, of course, not to be considered as limiting the invention. Numerous changes and modifications can be made with respect to embodiments of the invention.

In the context of embodiments of the subject invention and particularly with respect to the embodiments above and the claims appended hereto, the follow definitions are set forth.

A pretrained deep learning-based encoder for protein structure expression means a deep learning-based pretrained model for protein inverse folding. This type of model takes protein 3D structure as input, and creates as output protein sequences. By structure model means the model takes structure as input. In embodiments of the provided application, the latent representation of the structure models can be used as part of input to the model framework. The provided framework can fit any type of autoregressive protein sequence model.

The term, latent structure embeddings, means to make a more accurate mapping between protein structures and protein sequences, a provided deep neural network can present protein structures as a representation in a hidden space, where protein with similar structures are clustered. Thus, latent structure embeddings can be regarded as a better representation of protein structure for protein inverse folding.

A high dimensional space means, to achieve high expressiveness of protein, the latent embeddings are usually in spaces with higher dimensions than the original structure representation. In such embodiments it can be said that the latent space is of high dimension.

Autoregressive pretrained protein sequence decoder means using a type of model (e.g., autoregressive sequence model) that generates a next token based on the previous content of the sequence, which is known as next token prediction. Pretrained protein sequence model means that pretrained deep learning model that takes protein sequences as input and output protein sequences.

Based on protein language modeling techniques means following the approach of autoregressive generation of protein sequences.

Configured to provide automatic protein sequence generation means configured for autoregressive protein sequence generation.

A deep learning-based latent representation alignment model is one component of certain embodiments of the provided framework. Alignment means aligning latent structure embedding and latent sequence embedding of the same protein. In certain embodiments of the provided framework, the deep learning-based latent representation alignment model takes latent structure embedding as input, and outputs latent sequence embeddings that contain information of the input latent structure embeddings. In certain embodiments this is one of the novel designs of the provided framework.

Configured to provide connections between latent representations of protein sequences and protein structures means they are connected through the alignment model. The latent representations of protein structure is the input of alignment model. The model outputs the sequence latent representation that contains protein structure information. It serves as the guidance for protein sequence generation.

A compressed representation of a protein's structure means the output from a deep learning-based latent representation alignment model, which can be regarded as a compressed representation of a protein's structure.

An equal dimension graph encoder means the graph encoder comprises a deep neural network that takes graph representation of protein structure as input and outputs structure embeddings. Equal dimension means that the layers in the graph encoder have the same input and output dimension. Equal dimension is defined compared to protein structure encoders with decreasing dimension from input to the latent structure embeddings.

A multi layer perceptron (MLP) means a deep learning module that consists of multiple linear layers.

A structure encoder means a type of model that takes protein 3D structure as input, and output protein structure embeddings.

A sequence decoder means a type of model that is a component of a protein inverse folding model. The sequence decoder takes structure embeddings as input, decoding it into protein sequences as output.

With frozen parameters means the parameters of the model are not trainable. With unfrozen parameters means the parameters of the model are trainable.

Latent representation alignment means aligning latent structure embedding and latent sequence embedding of the same protein.

With regard to the pretrained deep learning-based encoder for protein structure expression and the autoregressive pretrained protein sequence decoder for autoregressive protein sequence generation, embodiments take protein structure as input, and generate protein sequences as output. The pretrained protein structure encoder maps protein structure into protein structure embeddings. Then the protein structure embeddings are fed into the alignment model, and the alignment model outputs latent embeddings that contains protein structure information as the latent sequence guidance embedding for protein sequence generation. The latent sequence guidance embeddings are fed into the autoregressive pretrained protein sequence decoder for protein sequences generation. So, the two models mentioned above are two components that work together in the framework of certain embodiments of the subject invention.

Embodiments use pretrained protein structure encoders. There are two main types of pretrained protein structure encoder: one is equal dimension graph encoder (e.g., the system and method known as PiFold), the other is unequal dimension graph decoder (e.g., the systems and methods known as ProteinMPNN and ESM-IF). Receiving load weights from the equal dimension graph encoder and the structure encoder means using the output structure embedding of these models.

Regarding making the model, (and Model Inference): Embodiments comprise a pretrained structure encoder, a deep learning-based latent representation alignment model, and an autoregressive protein sequence generation model. The provided framework takes protein structure as input, and generates protein sequences as output. The pretrained protein structure encoder maps protein structure into protein structure embeddings. Then the protein structure embeddings are fed into the alignment model, and the alignment model outputs latent embeddings that contains protein structure information as the latent sequence guidance embedding for protein sequence generation. The latent sequence guidance embeddings are fed into the autoregressive pretrained protein sequence decoder for protein sequences generation.

Regarding training the model: The training procedure is different from inference procedure. During training, for each protein, the protein sequence and the latent sequence guidance embeddings are concatenated as the input of pretrained protein sequence model. The loss is calculated by computing the cross-entropy between the output logits of pretrained protein sequence model and the ground-truth of protein sequence. Details of selected parameters are provided in Table 2.

TABLE 2
Details of selected parameters:

| | |
|---|---|
| Structure embedding dimension | 128, 512, 1152 |
| Sequence guidance embedding | 4096 |
| dimension | |
| Sequence guidance embedding length | [32, 64, 128, 256, 512] |
| Number of trainable parameters | 89M (M for Million) |
| Learning rate | 1e−4 |
| Number of warmup steps | 5000 |
| Training epoch | 100 |
| Batch size per device | [16, 32] |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### MATERIALS AND METHODS

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Creation and Testing of a Protein-Inverse Folding Method Based on Multi-Modal Pretrained Large Model According to an Embodiment of the Subject Invention

The provided architectural framework contains three main components, according to an embodiment of the subject invention: (1) the pre-trained structure encoder for protein inverse folding, (2) the pre-trained autoregressive protein LLM, and (3) the ProteinAligner.

The provided pretrained structure encoder utilizes structure encoders from existing protein inverse folding approaches, which can include (but are not limited to) ESM-IF [11], PiFold [9], and ProteinMPNN [3]. For this embodiment, we initialize the models with the officially released pretrained weights. For ProteinMPNN [3], nine weights including four vanilla model weights, two soluble model weights, and three Cα model weights. During implementation, we trained ProteinAligner with concatenated weights and vanilla model weights with 0.20 backbone noise.

The provided pretrained sequence auto-encoder (different from LM-Design [31]), generates protein sequences from scratch to ensure the access to novel samples with higher

diversity. We employ ProGen2 [20], a series of next-token-prediction autoregressive pretrained protein sequence decoders for protein language. We initialize models with different version, including ProGen2, including ProGen2 base (764M param), ProGen2-large (2.7B param), and ProGen2-Xlarge (6.4B param).

An embodiment comprising ProteinAligner provides efficient alignment between hidden protein sequence-structure representations using limited training data in a novel lightweight network. This network leverages the cross-attention technique for latent feature alignment and comprises four key components comprising the component layers for the deep learning-based alignment network: (1) a linear projection head for structure embeddings, (2) a cross-attention layer, (3) a positional embedding layer, and (4) an output projection layer mapping to language embedding. For module output stability, we include three normalization layers. All linear layers are initialized by trunc_normal_ with a standard deviation of 0.02 and zero bias. Layer normalization weights and biases are uniformly set to 1.0 and 0. To encapsulate structure embedding information, we initialize trainable structural queries of length 32 and incorporate a sine-cosine-based positional embedding with a stride length of 16, enhancing the model's sequential data comprehension and predictive performance.

For Structure Input, the raw information of protein structure is obtained from PDB files of open-source dataset CATH4.2 and CATH4.3. Structure inputs are selected based on different structure encoders and the details are shown in Table 3. We follow the same processing schedules for each approach.

TABLE 3
Details of structure information utilization and structure
embeddings of ESM-IF [11], ProteinMPNN [3], and PiFold [9].

| | | | Bond | Bond |
|---|---|---|---|---|
| Method | Dimension | Coordinate | Length | Angle |
| ESM-IF [11] | 512 | [C_{α}, N, C] | | |
| ProteinMPNN [3] | 128 | [C_{α}, C_{β}, N, C, O] | ✓ | |
| PiFold [9] | 128 | [C_{α}, N, C, O] | ✓ | ✓ |

To efficiently align structural information with sequence information, we provide learnable queries to dynamically express protein structure information. The queries are concatenated with input sequence embeddings, acting as structural prompts of sequence generation. Besides, embeddings of special tokens including <|structure|> and <|endofchunk|> are concatenated at the start and end of structure prompts.

We follow the same input format of ProGen2, protein sequences are encoded tokenizer of size 34. Starting symbol <|bos|> and ending symbol <|eos|> are concatenated at the start and end of the tokenized sequences, and all sequences are padded to the maximal length of 502.

The ProteinAligner exhibits direct and effective one-stage training for cross-modality alignment. In the training stage, all pretrained models are frozen, and the ProteinAligner is then initialized as detailed above.

We follow the same data split based on S40 version of CATH4.2 following PiFold [9] and ProteinMPNN [3], which brings train/valid/test dataset of amount 18024/608/1120. For VATH4.3 [22], we follow the same data split following ESM-IF [11]. Detailed data processing is shown in FIG. 1, Table 2, and Table 3. The ProteinAligner is trained from scratch by minimizing the cross entropy loss of protein-token level. The maximum learning rate is 1e⁻⁴, and we use a constant warm-up strategy for the first 5,000 steps. The whole training procedure was executed on 4 A100 GPUs with batch size 16 on each GPU.

The tested embodiment provides a novel protein design framework based on ProteinAligner to achieve generalized high quality protein inverse folding as well as diverse protein generation. Embodiments are shown to overcome the issue of limited training data in this field, presenting a novel approach to fine tune an autoregressive large autoregressive pretrained protein sequence decoder by aligning latent representations between protein structures and protein sequences. The provided framework enables more accurate sequence modeling based on prior knowledge of single-modality pretrained models. The provided framework obtains promising generalization of billion-level autoregressive protein LLMs. Extensive results demonstrate the effectiveness of aligner-guided tuning and the generative power of large pretrained models in sequence and structure modeling, achieving beneficial sequence designability of computational protein design. The provided framework offers a flexible and effective paradigm for protein cross-modality tasks.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Jean-Baptiste Alayrac, Jeff Donahue, Pauline Luc, Antoine Miech, lain Barr, Yana Hasson, Karel Lenc, Arthur Mensch, Katherine Millican, Malcolm Reynolds, et al. Flamingo: a visual language model for few-shot learning. Advances in Neural Information Processing Systems, 35:23716-23736, 2022.
[2] Sheng Chen, Zhe Sun, Lihua Lin, Zifeng Liu, Xun Liu, Yutian Chong, Yutong Lu, Huiying Zhao, and Yuedong Yang. To improve protein sequence profile prediction through image captioning on pairwise residue distance map. Journal of chemical information and modeling, 60(1):391-399, 2019.
[3] Justas Dauparas, Ivan Anishchenko, Nathaniel Bennett, Hua Bai, Robert J Ragotte, Lukas F Milles, Basile IM Wicky, Alexis Courbet, Rob J de Haas, Neville Bethel, et al. Robust deep learning-based protein sequence design using proteinmpnn. Science, 378(6615):49-56, 2022.
[4] Robbert J de Haas, Natalie Brunette, Alex Goodson, Justas Dauparas, Sue Y Yi, Erin C Yang, Quinton Dowling, Hannah Nguyen, Alex Kang, Asim K Bera, et al. Rapid and automated design of two-component protein nanomaterials using proteinmpnn. bioRxiv, pages 2023-08, 2023.
[5] Marianne Defresne, Sophie Barbe, and Thomas Schiex. Protein design with deep learning. International Journal of Molecular Sciences, 22(21): 11741, 2021.
[6] Noelia Ferruz and Birte Höcker. Controllable protein design with language models. Nature Machine Intelligence, 4(6):521-532, 2022.
[7] Noelia Ferruz, Steffen Schmidt, and Birte Höcker. A deep unsupervised language model for protein design. BioRxiv, pages 2022-03, 2022.
[8] Zhangyang Gao, Cheng Tan, and Stan Z Li. Alphadesign: A graph protein design method and benchmark on alphafolddb. arXiv preprint arXiv: 2202.01079, 2022.
[9] Zhangyang Gao, Cheng Tan, and Stan Z Li. Pifold: Toward effective and efficient protein inverse folding. arXiv preprint arXiv: 2209.12643, 2022.
[10] Juergen Haas, Rafal Gumienny, Alessandro Barbato, Flavio Ackermann, Gerardo Tauriello, Martino Bertoni, Gabriel Studer, Anna Smolinski, and Torsten Schwede. Introducing “best single template” models as reference baseline for the continuous automated model evaluation (cameo). Proteins, 87(12):1378-1387 December 2019. 31571280 [pmid].
[11] Chloe Hsu, Robert Verkuil, Jason Liu, Zeming Lin, Brian Hic, Tom Sercu, Adam Lerer, and Alexander Rives. Learning inverse folding from millions of predicted structures. In International Conference on Machine Learning, pages 8946-8970. PMLR, 2022.
[12] John Ingraham, Vikas Garg, Regina Barzilay, and Tommi Jaakkola. Generative models for graph-based protein design. Advances in neural information processing systems, 32, 2019.
[13] Bowen Jing, Stephan Eismann, Patricia Suriana, Raphael JL Townshend, and Ron Dror. Learning from protein structure with geometric vector perceptrons. arXiv preprint arXiv: 2009.01411, 2020.
[14] John Jumper, Richard Evans, Alexander Pritzel, Tim Green, Michael Figurnov, Olaf Ronneberger, Kathryn Tunyasuvunakool, Russ Bates, Augustin Židek, Anna Potapenko, et al. Highly accurate protein structure prediction with alphafold. Nature, 596(7873):583-589, 2021.
[15] Nobuyasu Koga, Rie Tatsumi-Koga, Gaohua Liu, Rong Xiao, Thomas B Acton, Gactano T Montelione, and David Baker. Principles for designing ideal protein structures. Nature, 491(7423):222-227, 2012.
[16] Junnan Li, Dongxu Li, Silvio Savarese, and Steven Hoi. Blip-2: Bootstrapping language image pre-training with frozen image encoders and large language models. arXiv preprint arXiv: 2301.12597, 2023.
[17] Haiyan Liu and Quan Chen. Computational protein design with data-driven approaches: Recent developments and perspectives. Wiley Interdisciplinary Reviews: Computational Molecular Science, 13(3):e1646, 2023.
[18] Haotian Liu, Chunyuan Li, Qingyang Wu, and Yong Jac Lee. Visual instruction tuning. arXiv preprint arXiv: 2304.08485, 2023.
[19] Ali Madani, Bryan McCann, Nikhil Naik, Nitish Shirish Keskar, Namrata Anand, Raphael R Eguchi, Po-Ssu Huang, and Richard Socher. Progen: Language modeling for protein generation. bioRxiv, pages 2020-03, 2020.
[20] Erik Nijkamp, Jeffrey Ruffolo, Eli N Weinstein, Nikhil Naik, and Ali Madani. Progen2: exploring the boundaries of protein language models. arXiv preprint arXiv: 2206.13517, 2022.
[21] James O'Connell, Zhixiu Li, Jack Hanson, Rhys Heffernan, James Lyons, Kuldip Paliwal, Abdollah Dehzangi, Yuedong Yang, and Yaoqi Zhou. Spin2: Predicting sequence profiles from protein structures using deep neural networks. Proteins: Structure, Function, and Bioinformatics, 86(6):629-633, 2018.
[22] Christine A Orengo, Alex D Michie, Susan Jones, David T Jones, Mark B Swindells, and Janet M Thornton. Cath-a hierarchic classification of protein domain structures. Structure, 5(8):1093-1109, 1997.
[23] Robin Pearce and Yang Zhang. Deep learning techniques have significantly impacted protein structure prediction and protein design. Current opinion in structural biology, 68:194-207, 2021.
[24] Alexander Rives, Joshua Meier, Tom Sercu, Siddharth Goyal, Zeming Lin, Jason Liu, Demi Guo, Myle Ott, C Lawrence Zitnick, Jerry Ma, et al. Biological structure and function emerge from scaling unsupervised learning to 250 million protein sequences. Proceedings of the National Academy of Sciences, 118(15):e2016239118, 2021.
[25] Gabriel J Rocklin, Tamuka M Chidyausiku, Inna Goreshnik, Alex Ford, Scott Houliston, Alexander Lemak, Lauren Carter, Rashmi Ravichandran, Vikram K Mulligan, Aaron Chevalier, et al. Global analysis of protein folding using massively parallel design, synthesis, and testing. Science, 357(6347):168-175, 2017.
[26] Michael Schauperl and Rajiah Aldrin Denny. Ai-based protein structure prediction in drug discovery: impacts and challenges. Journal of Chemical Information and Modeling, 62(13):3142-3156, 2022.
[27] Paweł S′ledz' and Amedeo Caflisch. Protein structure-based drug design: from docking to molecular dynamics. Current opinion in structural biology, 48:93-102, 2018.
[28] Lorillee Tallorin, JiaLei Wang, Woojoo E Kim, Swagat Sahu, Nicolas M Kosa, Pu Yang, Matthew Thompson, Michael K Gilson, Peter I Frazier, Michael D Burkart, et al. Discovering de novo peptide substrates for enzymes using machine learning. Nature communications, 9(1):5253, 2018.
[29] Cheng Tan, Zhangyang Gao, Jun Xia, Bozhen Hu, and Stan Z Li. Global-context aware generative protein design. In ICASSP 2023-2023 IEEE International Conference on Acoustics, Speech and Signal Processing (ICASSP), pages 1-5. IEEE, 2023.
[30] Michel van Kempen, Stephanie S Kim, Charlotte Tumescheit, Milot Mirdita, Cameron LM Gilchrist, Johannes Söding, and Martin Steinegger. Foldseek: fast and accurate protein structure search. Biorxiv, pages 2022-02, 2022.
[31] Zaixiang Zheng, Yifan Deng, Dongyu Xue, Yi Zhou, Fei Ye, and Quanquan Gu. Structure informed language models are protein designers. bioRxiv, pages 2023-02, 2023.

We claim:

1. A protein design system (10) for protein inverse folding and computational protein sequence design, the system comprising:
a multi-modal alignment module (100) comprising:
a pretrained deep learning-based encoder (102) configured for protein structure latent expression in a high dimensional space,
a ProteinAligner (103) comprising a deep learning-based latent representation alignment model; and
an autoregressive pretrained protein sequence decoder (104) configured for protein sequence expression.

2. The system according to claim 1, further comprising:
an autoregressive graph-based generation module (200); and
a one-shot graph-based generator (300).

3. The system according to claim 2, wherein the autoregressive graph-based generation module (200) comprises:
a protein structure encoder (202), and
a protein sequence decoder (203).

4. The system according to claim 3, wherein the one-shot graph-based generator (300) comprises:
an equal-dimension graph encoder (302), and
a multi layer perceptron (MLP) (303).

5. The system according to claim 4, wherein the pretrained deep learning-based encoder (102) comprises one or more pretrained autoregressive encoder weights (240) derived from the protein structure encoder (202) and one or more pretrained one-shot encoder weights (340) derived from the equal-dimension graph encoder (302).

6. The system according to claim 5, wherein the one or more pretrained autoregressive encoder weights (240) are derived from the protein structure encoder (202) at least in part by a supervised machine learning process wherein the protein structure encoder (202) is unfrozen.

7. The system according to claim 6, wherein the one or more pretrained one-shot encoder weights (340) are derived from the equal-dimension graph encoder (302) at least in part by a supervised machine learning process wherein the equal-dimension graph encoder (302) is unfrozen.

8. The system according to claim 6, wherein the one or more pretrained autoregressive encoder weights (240) are derived from the protein structure encoder (202) at least in part by a supervised machine learning process wherein the protein sequence decoder (203) is unfrozen.

9. The system according to claim 7, wherein the one or more pretrained one-shot encoder weights (340) are derived from the equal-dimension graph encoder (302) at least in part by a supervised machine learning process wherein the MLP (303) is unfrozen.

10. The system according to claim 5, wherein the deep learning-based latent representation alignment model of the ProteinAligner (103) is trained at least in part by a supervised machine learning process wherein the ProteinAligner (103) is unfrozen.

11. A method for making a protein design system (10) for protein inverse folding and computational protein sequence design, the method comprising:
providing a multi-modal alignment module (100) comprising a pretrained deep learning-based encoder (102), a ProteinAligner (103), and an autoregressive pretrained protein sequence decoder (104);
providing an autoregressive graph-based generation module (200) comprising a protein structure encoder (202), and a protein sequence decoder (203);
providing a one-shot graph-based generator (300) comprising an equal-dimension graph encoder (302), and a multi layer perceptron (MLP) (303);
training the autoregressive graph-based generation module (200) to produce one or more pretrained autoregressive encoder weights (240) from the protein structure encoder (202); and
inserting the one or more pretrained autoregressive encoder weights (240) into the pretrained deep learning-based encoder (102).

12. The method according to claim 11, further comprising:
training the one-shot graph-based generator (300) to produce the one or more pretrained one-shot encoder weights (340) from the equal-dimension graph encoder 302; and
inserting the one or more pretrained one-shot encoder weights (340) into the pretrained deep learning-based encoder (102).

13. The method according to claim 12, wherein the training the autoregressive graph-based generation module (200) to produce the one or more pretrained autoregressive encoder weights (240) from the protein structure encoder (202) comprises training the autoregressive graph-based generation module (200) while both the protein structure encoder (202) and the protein sequence decoder (203) are unfrozen.

14. The method according to claim 13, wherein the training the one-shot graph-based generator (300) to produce the one or more pretrained one-shot encoder weights (340) from the equal-dimension graph encoder (302) comprises training the one-shot graph-based generator (300) while both the equal-dimension graph encoder (302) and the multi layer perceptron (MLP) (303) are unfrozen.

15. The method according to claim 14, further comprising training the multi-modal alignment module (100) while the pretrained deep learning-based encoder (102) is frozen with the one or more pretrained autoregressive encoder weights (240) and the one or more pretrained one-shot encoder weights (340).

16. The method according to claim 15, wherein the training the multi-modal alignment module (100) while the pretrained deep learning-based encoder (102) is frozen with the one or more pretrained autoregressive encoder weights (240) and the one or more pretrained one-shot encoder weights (340) comprises training the multi-modal alignment module (100) while the autoregressive pretrained protein sequence decoder (104) is also frozen.

17. The method according to claim 16, wherein the training the multi-modal alignment module (100) while the pretrained deep learning-based encoder (102) is frozen with the one or more pretrained autoregressive encoder weights (240) and the one or more pretrained one-shot encoder weights (340) and the autoregressive pretrained protein sequence decoder (104) is also frozen further comprises training the multi-modal alignment module (100) while the ProteinAligner (103) is unfrozen.

18. A protein design system (10) for protein inverse folding and computational protein sequence design, the system comprising:
a multi-modal alignment module (100) comprising:
a pretrained deep learning-based encoder (102) configured for protein structure latent expression in a high dimensional space,
a ProteinAligner (103) comprising a deep learning-based latent representation alignment model; and
an autoregressive pretrained protein sequence decoder configured for protein sequence expression;
an autoregressive graph-based generation module (200) comprising:
a protein structure encoder (202), and
a protein sequence decoder (203); and
a one-shot graph-based generator (300) comprising:
an equal-dimension graph encoder (302), and
a multi layer perceptron (MLP) (303).

19. The system according to claim 18, wherein:
the pretrained deep learning-based encoder (102) comprises one or more pretrained autoregressive encoder weights (240) derived from the protein structure encoder (202) and one or more pretrained one-shot encoder weights (340) derived from the equal-dimension graph encoder (302);
the one or more pretrained autoregressive encoder weights (240) are derived from the protein structure encoder (202) at least in part by a supervised machine learning process wherein the protein structure encoder (202) is unfrozen;
the one or more pretrained one-shot encoder weights (340) are derived from the equal-dimension graph encoder (302) at least in part by a supervised machine learning process wherein the equal-dimension graph encoder (302) is unfrozen;
the one or more pretrained autoregressive encoder weights (240) are derived from the protein structure encoder (202) at least in part by a supervised machine learning process wherein the protein sequence decoder (203) is unfrozen; and
the one or more pretrained one-shot encoder weights (340) are derived from the equal-dimension graph encoder (302) at least in part by a supervised machine learning process wherein the MLP (303) is unfrozen.

20. The system according to claim 19, wherein the deep learning-based latent representation alignment model of the ProteinAligner (103) is trained at least in part by a supervised machine learning process wherein the ProteinAligner (103) is unfrozen.
